(19) United States
(12) Patent Application Publication
(10) Pub. No.: US 2023/0172756 A1
(43) Pub. Date: Jun. 8, 2023

(54) METHOD FOR CONTROLLING A LASER OF A LASER DEVICE, METHOD FOR PERFORMING A SURGICAL PROCEDURE, LASER DEVICE, COMPUTER PROGRAM AND COMPUTER-READABLE MEDIUM

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/061,728

(22) Filed: Dec. 5, 2022

(30) Foreign Application Priority Data
Dec. 8, 2021 (DE) 10 2021 132 290.1

(51) Int. Cl. A61F 9/008; A61F 2/14

(52) U.S. Cl. CPC A61F 9/00829; A61F 9/00834; A61F 2/145; A61F 2009/00872

(57) ABSTRACT

The invention relates to a method for controlling a laser (12) of a laser device (10) and/or to a method for performing a surgical procedure comprising at least the steps of:
generating laser pulses (40) with a first energy density (42) below a photodisruption regime of a polymer material (26) of a region (16) of an optical element;
irradiating a core region (30) with the laser pulses (40), wherein a refractive index of the polymer material (26) changes depending thereon;
generating first irradiation lines (34) within the core region (30) and generating a first optical correction (44) in the core region (30);
generating laser pulses (40) with a second energy density (46) below a photodisruption regime;
irradiating an edge region (36) with the laser pulses (40), wherein the refractive index of the polymer material (26) changes depending thereon; and
generating second irradiation lines (38) within the edge region (36) and generating a second optical correction (48) in the edge region (36). Further, the invention relates to a laser device (10), to a computer program as well as to a computer-readable medium.

### FIELD

The invention relates to a method for controlling a laser of a laser device as well as to a method for performing a surgical procedure. Further, the invention relates to a laser device, to a computer program as well as to a computer-readable medium.

### BACKGROUND

Opacities and scars within the cornea, which can arise by inflammations, injuries or native diseases, impair the sight. In particular in case that these pathological and/or unnaturally altered regions of the cornea are located in the axis of vision of the eye, a clear sight is considerably disturbed. Furthermore, further visual disorders, such as for example a reduced visual acuity or corneal curvatures, can also impair the sight. Hereto, different laser methods by means of corresponding laser devices are given from the prior art, which separate a volume body from the cornea and thus can improve the sight for a patient. For example, photodisruptive and ablative methods are known hereto, which generate corresponding interfaces via laser pulses, and a volume body can for example be removed from the cornea thereby, whereby the injured or pathological region can be changed such that the sight is again improved.

Further, methods are already known from the prior art, in which a focused femtosecond laser with low energy and high repetition rate is employed to change a refractive index of transparent materials and tissues, such as for example a cornea, lens, contact lenses and artificial eye lenses, in non-surgical manner and thus to change the light refraction characteristics thereof. This method is in particular also referred to as LIRIC (Laser-Induced Refractive Index Change). In living tissues, the method does not initiate wound healing or scar formation.

According to the known LIRIC methods, cylindrical and spiral paths are used, respectively, to provide the phase-interleaved optical corrections. Therein, the result of the treatment is dependent on the speed of the output of the laser pulses, wherein a consistent speed is herein preset from the prior art. In particular in the concentric paths, however, this is difficult to solve since in particular depending on the position of the laser pulses, the laser speed then cannot be maintained anymore for example in the center of the circular path, or corresponding corrections either can no longer be performed at the edge of the treatment region due to limitations of the laser device with respect to the energy provision.

### SUMMARY

Therefore, it is the object of the present invention to provide a laser device, a computer program and a computer-readable medium, by means of which the disadvantages of the prior art are overcome and in particular an improved treatment of a polymer material, in particular of a cornea, can be realized.

This object is solved by a method, a laser device, a computer program as well as a computer-readable medium according to the independent claims. Advantageous forms of configuration with convenient developments of the invention are specified in the respective dependent claims, wherein advantageous configurations of the method are to be regarded as advantageous configurations of the laser device, of the computer program and of the computer-readable medium and vice versa.

An aspect of the invention relates to a method for controlling a laser of a laser device as well as to a method for performing a surgical procedure. Generating a plurality of first laser pulses with a first energy density within a preset energy range and below a photodisruption regime of a polymer material of a region of an optical element is effected. A core region of the region is irradiated with the first laser pulses, wherein a refractive index of the polymer material changes at each irradiation spot irradiated with the first laser pulses depending thereon. A plurality of first irradiation lines is generated within the core region by means of a plurality of irradiation spots and a first optical correction is generated in the core region thereby. Generating a plurality of second laser pulses with a second energy density within the energy range and below a photodisruption regime of the polymer material of the region of the optical element is effected, wherein the second energy density is different from the first energy density. The edge region of the region is irradiated with the second laser pulses, which surrounds the core region at least in certain areas, wherein the refractive index of the polymer material changes at each of the irradiation spots irradiated with the second laser pulses depending thereon. A plurality of second irradiation lines is generated within the edge region by means of the plurality of irradiation spots and generation of a second optical correction different from the first optical correction is thereby effected in the edge region.

Thus, it is in particular proposed that the region to be treated is divided into the core region and into the edge region. In the core region, the irradiation is effected with the first energy density and in the edge region with the second energy density. In particular, the energy dose is to be understood by energy density, that is for example the energy per area unit. Thus, it can be the energy of a laser pulse per area, but also the energy of multiple pulses within a “scan part” per area of the scan path, thus of the irradiation line. In particular, the energy density can also be regarded as power density, that is energy per time unit and per area unit. Further, the so-called “fluence”, that is energy per volume unit, can also be regarded as the energy density. Therein, the energy density can be described via the density of the irradiation lines, the so-called interline distance, or else via the density of the irradiation spots within the lines, thus the density of the irradiation spots, which is also referred to as interspot distance. Further, the energy of the emitted laser pulses for different lines can also be understood by energy density.

The energy range describes a range, in which the energy can be adjusted by a user of the laser device and the method according to the invention can be performed. In particular, the energy range is upwards limited by the energy for the photodisruption regime, thus from which energy photodisruption bubbles, which can also be referred to as cavitation bubbles, arise, and downwards limited by that energy, at which the refractive index in the polymer material still changes. Thus, the energy range is in particular dependent on the polymer material itself.

In particular, the polymer material is a human or animal cornea and/or a lens of an eye. Further, it is also to be mentioned at this place that the method can also be applied to artificial contact lenses or artificial intraocular lenses. Thus, the polymer material is in particular for example a biopolymer material, which is synthesized in a cell of a living being, for example in the form of polysaccharides, proteins, nucleic acids or the like. However, artificial polymer materials such as for example collagen can further also be correspondingly changed with the method according to the invention. For example, the cornea and the lens of the eye, respectively, can then be considered as the biopolymer material, while contact lenses or intraocular lenses can for example be regarded as collagen.

Then, the optical element can for example be an eye or a vitreous body. The region in turn describes that region within the polymer material, which for example encompasses the pathological region and/or region to be treated.

Thus, the laser pulses with the predefined energy or energy density are in particular generated such that they are generated below the photodisruption regime, which means that photodisruption bubbles are not generated within the cornea, whereby only the refractive index is changed within the irradiation spots, whereby, after the treatment, light beams are in turn refracted in the region differently than before, which results in a correction on the optical element.

Thus, it is further preferably provided that a so-called phase wrapping is performed within the region. In particular, a technical boundary, in particular in the edge region, which can also be referred to as periphery, is achieved in the so-called phase wrapping, in which the resolution of the laser device cannot allow “finer” irradiation lines anymore. Now, it is provided according to the invention that a progressive decrease of the irradiation line profile, in particular in the edge region, can be performed, wherein it can in particular be performed in a so-called transitional zone. A presbyopic correction, thus a correction of the presbyopia, can also be performed with the method.

According to an advantageous form of configuration, an optical second correction in the edge region is generated inferior than the optical first correction. Thus, it is in particular provided that different corrections are performed in the edge region and in the core region. Caused by the limitation of the laser device, it is for example no longer allowed to perform the corresponding correction in the edge region as in the core region. However, it is now in particular allowed that an inferior correction is performed in the edge region, which can result in an improvement for example of the vision. Thus, the values of the corrections preferably differ in their order of magnitude, but not in their sign. For example, the first correction can be at five diopters and the second correction at less than five diopters, for example at four diopters or at two diopters. This is to be regarded purely exemplarily and not as conclusive at all.

Further, it has proven advantageous if the first irradiation lines and/or the second irradiation lines are substantially annularly generated in the region. Thus, a kind of Fresnel lens can in particular be provided, wherein both the first irradiation lines in the core region and the second irradiation lines in the edge region are thus annularly formed.

It is further advantageous if the first irradiation lines and/or the second irradiation lines are generated concentrically to each other. Thus, the first irradiation lines and/or the second irradiation lines in particular form concentric circles to each other. In particular, the concentric circles have a lower radius in the core region than the concentric circles in the edge region. Thus, a reliable correction of the polymer material can be realized.

In a further advantageous form of configuration, a transition from the core region to the edge region is preset depending on at least one parameter limiting the laser device. In particular if the laser device has reached a corresponding limit with respect to the potentially achievable energy density, in particular with respect to the laser pulses to be emitted, or irradiation spot distances, thus, it is transitioned into the edge region to perform a further correction with a lower energy density there. Thus, a corresponding correction can also be performed in the edge region, which can result in an improvement of the sight restriction.

In a further advantageous form of configuration, the edge region is used as the transitional zone from the region to be treated to a region not to be treated. The transitional zone can also be referred to as transition zone (TZ). Thus, an improved transition between the region to be treated and the region not to be treated can be generated, whereby an improvement of the visual impairment can be realized.

It is further advantageous if a predefined correction of the optical element is performed in the core region. In particular, the predefined correction can for example be preset due to pachymetric data of the eye. In particular, the predefined correction allows the improved eyesight. In the edge region, a kind of transitional region can then in turn be provided, which does not have the predefined correction, but still can realize an improvement of the visual acuity also in the edge region and thus an improved transition to the region not to be treated.

According to a further advantageous form of configuration, at least the first energy density is adjusted depending on the respective distance of the irradiation spots to each other and/or depending on respective distances of the first irradiation lines to each other and/or depending on a laser pulse energy of the respective laser pulses. Thus, the energy density is in particular not only dependent on an individual pulse, but can be considered depending on the energy dose, thus the energy per area unit. Thus, the energy of a laser pulse per area, but also the energy of multiple pulses within the area can influence the energy density. Further, the energy, with which the laser pulses are generated, can also correspondingly influence the energy density. Furthermore, the energy density can also be understood as a type of power density, that is the energy per time unit and per area unit. Further, the energy density can also be understood as the so-called fluence, that is the energy per volume unit. Thus, the energy density can be influenced in different manners.

Further, it has proven advantageous if an axicon shape of the optical region is generated by means of the method. Thereby, a special, conically formed region can for example be generated. Axicons are conical lenses, which generate an annular beam profile. An axicon maps a point source to a line along the optical axis or transforms a laser beam into a ring, whereby optical corrections can be performed in the region. The axicon shape can for example be generated based on different heights of the irradiation lines. For example, a height of the irradiation lines can decrease from the center of the core region towards the edge region.

It is also advantageous if at least a height of the second irradiation line is generated differently than the height of the first irradiation line and/or a distance between the second irradiation lines is generated differently than a distance of the first irradiation lines and/or the laser pulses are generated with a lower predefined second energy for the second irradiation lines than the laser pulses with the predefined first energy for the first irradiation lines for generating the second optical correction. Thus, an optical correction can in particular be performed with different interspot distances, interline distances as well as different energy densities of the laser pulses itself. Further, a corresponding height of the irradiation lines can also be different. Thus, different optical corrections can be performed in the core region and in the edge region.

In a further advantageous form of configuration, different second corrections are generated in the edge region from an inner edge, which faces the core region, to an outer edge, which faces away from the core region. In particular, the different second corrections are dependent on corresponding limitations of the laser device. Thus, a transitional zone can in particular be provided, which has different second corrections, such that a smooth transition from the core region to the outermost edge of the edge region can be generated, whereby an improved optical correction can be realized.

Again advantageously, an inferior correction is generated at the outer edge of the edge region than at the inner edge of the edge region. Thus, a smooth transition from the core region via the edge region towards the region not to be treated can be generated.

It has further proven advantageous if the laser pulses are emitted in a wavelength range between 200 nm and 2 μm, in particular between 400 nm and 1450 nm, at a respective pulse duration between 1 fs and 1 ps, in particular between 10 fs and 100 fs, and a repetition frequency of greater than 10 kHz, in particular between 1 MHz and 100 MHz. Thereby, the laser pulses can in particular be generated below the photodisruption regime, which only results in a change of the refractive index. Thereby, the method and in particular the change of the refractive index can be reliably performed without performing an invasive intervention in the cornea.

In a further advantageous form of configuration, topographic and/or pachymetric and/or morphologic data of the optical element, in particular of the eye, in particular of the cornea and/or the lens, is taken into account in controlling the laser. In particular, this data can for example already be determined before a treatment. Based on this data, the treatment can then be reliably performed.

A second aspect of the invention relates to a laser device with at least one eye surgical laser and with at least one control device for the laser or lasers, which is formed to perform the steps of the method according to the first aspect.

Preferably, the laser device is formed as a rotation scanner and hereto for example comprises a beam deflection device. Further, the laser device is preferably formed as an eye surgical treatment apparatus.

In an advantageous form of configuration of the laser device, the laser device comprises a storage device for at least temporary storage of at least one control dataset, wherein the control dataset or datasets include(s) control data of individual laser pulses on or in the optical element, and includes at least one beam deflection device for beam guidance and/or beam shaping and/or beam deflection and/or beam focusing of a laser beam of the laser. Therein, the mentioned control datasets are usually generated based on a measured topography and/or pachymetry and/or morphology of the optical element to be treated, in particular the cornea or lens to be treated of the pathologically and/or unnaturally altered region within the optical element.

Therein, it can be provided that the laser device comprises a single storage device and a single control device. Alternatively, it can be provided that different storage devices and control devices are formed within the laser device to perform a corresponding control of the laser.

Further features and the advantages thereof can be taken from the descriptions of the first inventive aspect, wherein advantageous configurations of the first inventive aspect are to be regarded as advantageous configurations of the respectively other inventive aspect.

A third aspect relates to a computer program including commands, which cause the laser device according to the second inventive aspect to execute the method steps according to the first inventive aspect. A fourth aspect of the invention relates to a computer-readable medium, on which the computer program according to the third inventive aspect is stored.

Further features and the advantages thereof can be taken from the descriptions of the first and second inventive aspects, wherein advantageous configurations of each inventive aspect are to be regarded as advantageous configurations of the respectively other inventive aspect.

### BRIEF DESCRIPTION OF DRAWINGS

Further features are apparent from the claims, the figures and the description of figures. The features and feature combinations mentioned above in the description as well as the features and feature combinations mentioned below in the description of figures and/or shown in the figures alone are usable not only in the respectively specified combination, but also in other combinations without departing from the scope of the invention. Thus, implementations are also to be considered as encompassed and disclosed by the invention, which are not explicitly shown in the figures and explained, but arise from and can be generated by separated feature combinations from the explained implementations. Implementations and feature combinations are also to be considered as disclosed, which thus do not comprise all of the features of an originally formulated independent claim. Moreover, implementations and feature combinations are to be considered as disclosed, in particular by the implementations set out above, which extend beyond or deviate from the feature combinations set out in the relations of the claims.

FIG. 1 shows a schematic view of an embodiment of a laser device.

FIG. 2 shows a schematic top view to an optical element.

FIG. 3 shows a schematic sectional view to an optical element.

In the figures, identical or functionally identical elements are provided with the same reference characters.

### DETAILED DESCRIPTION

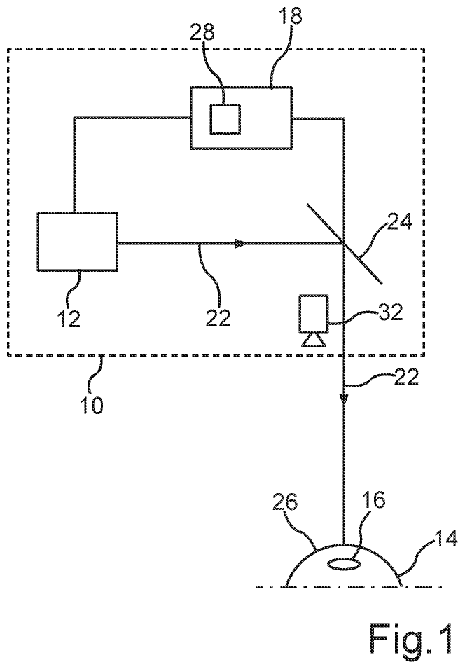

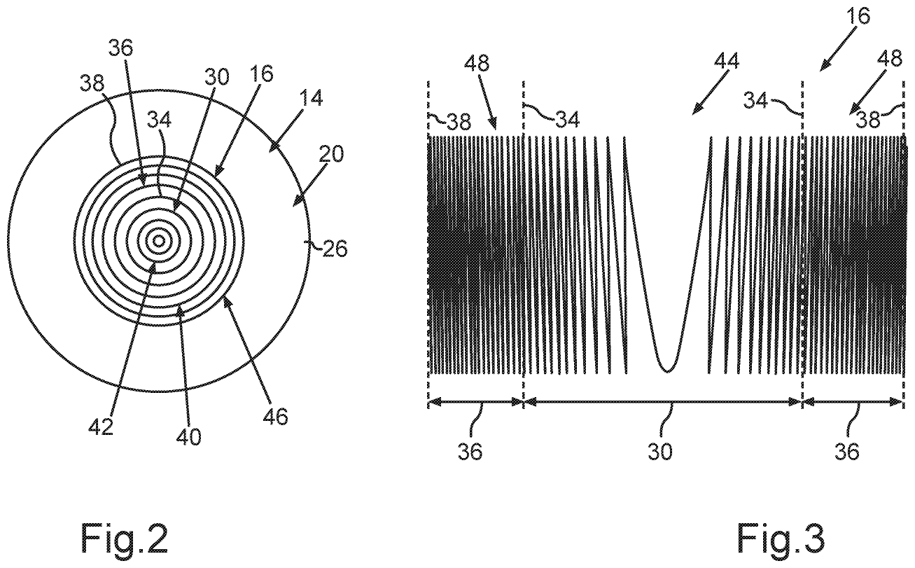

FIG. 1 shows a schematic representation of a laser device 10 with a laser 12 for example for the treatment of a patient, in particular for the treatment of an eye 14 of a patient, wherein the eye 14 is also referred to as optical element below. Thus, the laser device 10 is presently formed as an eye surgical treatment apparatus. One recognizes that a control device 18 for the laser 12 is formed besides the laser 12. This form of configuration with a control device 18 is to be purely exemplarily regarded. It can be provided that the laser device 10 also comprises a plurality of, in particular more than two, control devices 18. For example, the control device 18 can emit pulsed laser pulses 40 (see FIG. 2) in a predefined pattern into the eye 14, for example into a region 16, wherein the position of the region 16 is selected in this embodiment such that a pathological and/or unnaturally altered region within a stroma of the eye 14 is enclosed. Thus, the region 16 is a region 16 to be treated.

Furthermore, one recognizes that the laser beam 22 generated by the laser 12 is deflected towards the eye 14 by means of a beam deflection device 24 such as for example a scanner, in particular a so-called rotation scanner. The beam deflection device 24 is also controlled by the control device 18 to for example generate irradiation lines 34, 38 (see FIG. 2). The beam deflection device 24 can for example comprise one or also two mirrors, which are formed for deflecting the impinging laser beam 22.

In the present embodiment, the illustrated laser 12 is a laser 12, which emits the laser pulses 40 in a wavelength range between 200 nm and 2 μm, in particular between 400 nm and 1450 nm, at a respective pulse duration between 1 fs and 1 ps, in particular between 10 fs and 100 fs, and a repetition frequency of greater than 10 kHz, in particular between 1 MHz and 100 MHz. Thereby, the laser pulses 40 can in particular be generated below the photodisruption regime, which only results in a change of the refractive index. Thereby, the method and in particular the change of the refractive index can be reliably performed without performing an invasive intervention for example in a cornea.

In addition, the control device 18 comprises a storage device 28 for at least temporary storage of at least one control dataset, wherein the control dataset or datasets include(s) control data for positioning and/or for focusing individual laser pulses 34 in or on the eye 14. The position data and/or focusing data of the individual laser pulses 40 are generated based on a previously measured topography and/or pachymetry and/or the morphology of the eye 14 and, for example, the pathological and/or unnaturally altered region within the stroma of the eye 14.

FIG. 2 shows a schematic top view to an eye 14. In particular, FIG. 2 shows the region 16, which is to be treated, as well as a region 20 not to be treated. The region 16 to be treated is divided into a core region 30, which is bounded by a first irradiation line 34, as well as into an edge region 36, which is bounded at an outer edge by a second irradiation line 38 as well as at the inner edge by the first irradiation line 34.

In the method according to the invention, it is provided that a plurality of first laser pulses 40 with a first energy density 42 within a preset energy range and below a photodisruption regime of a polymer material 26 of the region 16 of the optical element is generated. Irradiating the core region 30 with the first laser pulses 40 is effected, wherein a refractive index of the polymer material 26 changes at each irradiation spot irradiated with the first laser pulses 40 depending thereon. A plurality of first irradiation lines 34 is generated within the core region 30 by means of a plurality of irradiation spots, whereby a first optical correction 44 (see FIG. 3) is generated in the core region 30. Generating a plurality of second laser pulses 40 with a second energy density 46 within the energy range and below a photodisruption regime of the polymer material 26 of the region 16 of the optical element is effected, wherein the second energy density 46 is different from the first energy density 42. The edge region 36 is irradiated with the second laser pulses 40, wherein the edge region 36 surrounds the core region 30 at least in certain areas, and wherein the refractive index of the polymer material 26 changes at each irradiation spot irradiated with the second laser pulses 40 depending thereon. A plurality of second irradiation lines 38 is generated within the edge region 36, whereby a second correction 48 different from the first optical correction 44 is generated in the edge region 36.

Therein, it is in particular provided that the second optical correction 48 in the edge region 36 is generated inferior than the optical first correction 44 in the core region 30. Further, it is in particular shown that the first irradiation lines 34 and/or the second irradiation lines 38 are substantially annularly generated in the region 16, wherein it is in particular provided therein that the first irradiation lines 34 and/or the second irradiation lines 38 are generated concentrically to each other.

Further, it is in particular provided that a predefined correction of the optical element, in particular of the eye 14, is performed at least in the core region 30. Further, FIG. 2 shows that different second corrections 48 are generated in the edge region 36 from an inner edge, which is presently bounded by the first irradiation line 34 and which faces the core region 30, to an outer edge, which is presently bounded by the second irradiation line 38 and faces away from the core region 30. Therein, an inferior correction can in particular be generated at the outer edge of the edge region 36 than at the inner edge of the edge region 36.

FIG. 3 shows a schematic side view and sectional view, respectively, of the region 16. Presently, a type of sectional view of the region 16 shown in FIG. 2 is in particular shown.

FIG. 3 shows that a transition from the core region 30 to the edge region 36 is preset depending on at least one parameter limiting the laser device 10. In particular if the laser device 10 has reached a corresponding limit with respect to the energy density 42, 46, thus, it is transitioned into the edge region 36, to perform a further correction, in particular the second optical correction 48, with a lower energy density 42, 46 there. Thus, a corresponding correction can also be performed in the edge region 36, which can result in an improvement of the sight restriction.

Therein, it can be provided that the edge region 36 is for example used as a transitional zone from the region 16 to be treated to the region 20 not to be treated.

Further, FIG. 3 shows that at least the first energy density 42 is adjusted depending on the respective distance of the irradiation spots to each other and/or depending on respective distances of the first irradiation lines 34 to each other and/or depending on a laser pulse energy of the respective laser pulses 40. Further, at least a height of the second irradiation line 38 can be generated differently than the height of the first irradiation line 34 and/or a distance between the second irradiation lines 38 can be generated differently than a distance of the first irradiation line 34 and/or the laser pulses 40 can be generated with a lower predefined second energy for the second irradiation lines 38 than the laser pulses 40 with the predefined first energy for the first irradiation lines 34 for generating the second optical correction 48.

FIG. 3 in particular shows that the heights of the first irradiation line 34 and the second irradiation line 38 are substantially identical. Alternatively, it can also be provided that an axicon shape of the optical region 16 is generated by means of the method.

1. A method for controlling a laser of a laser device comprising at least the following steps:
generating a plurality of first laser pulses with a first energy density within a preset energy range and below a photodisruption regime of a polymer material of a region of an optical element;
irradiating a core region of the region with the first laser pulses, wherein a refractive index of the polymer material changes at each irradiation spot irradiated with the first laser pulses depending thereon;
generating a plurality of first irradiation lines within the core region by means of a plurality of irradiation spots and thereby generating a first optical correction in the core region;
generating a plurality of second laser pulses with a second energy density within the energy range and below a photodisruption regime of the polymer material of the region of the optical element, wherein the second energy density is different from the first energy density;
irradiating an edge region of the region, which surrounds the core region at least in certain areas, with the second laser pulses, wherein the refractive index of the polymer material changes at each irradiation spot irradiated with the second laser pulses depending thereon; and
generating a plurality of second irradiation lines within the edge region by means of a plurality of irradiation spots and thereby generating a second optical correction different from the first optical correction in the edge region.

2. The method according to claim 1, wherein the second optical correction in the edge region is generated inferior than the first optical correction in the core region.

3. The method according to claim 1, wherein the first irradiation lines and/or the second irradiation lines are substantially annularly generated in the region.

4. The method according to claim 3, wherein the first irradiation lines and/or the second irradiation lines are generated concentrically to each other.

5. The method according to claim 1, wherein a transition from the core region to the edge region is preset depending on at least one parameter limiting the laser device.

6. The method according to claim 1, wherein the edge region is used as a transitional zone from the region to be treated to a region not to be treated.

7. The method according to claim 1, wherein a predefined correction of the optical element is performed in the core region.

8. The method according to claim 1, wherein at least the first energy density is adjusted depending on a respective distance of the irradiation spots to each other and/or depending on respective distances of the first irradiation lines to each other and/or depending on a laser pulse energy of the respective laser pulses.

9. The method according to claim 1, wherein an axicon shape of the optical region is generated by means of the method.

10. The method according to claim 1, wherein at least a height of the second irradiation line is generated differently than the height of the first irradiation lines and/or a distance between the second irradiation lines is generated differently than a distance of the first irradiation lines and/or the laser pulses are generated with a lower predefined second energy for the second irradiation lines than the laser pulses with the predefined first energy for the first irradiation lines for generating the second optical correction.

11. The method according to claim 1, wherein different second corrections are generated in the edge region from an inner edge, which faces the core region, to an outer edge, which faces away from the core region.

12. The method according to claim 11, wherein at the outer edge of the edge region, an inferior correction is generated than at the inner edge of the edge region.

13. The method according to claim 1, wherein the laser pulses are emitted in a wavelength range between 200 nm and 2 μm, in particular between 400 nm and 1450 nm, at a respective pulse duration between 1 fs and 1 ps, in particular between 10 fs and 100 fs, and a repetition frequency of greater than 10 kHz, in particular between 1 MHz and 100 MHz.

14. The method according to claim 1, wherein the control of the laser is effected such that topographic and/or pachymetric and/or morphologic data of a cornea as the polymer material is taken into account.

15. A laser device with at least one eye surgical laser and with at least one control device for the laser or lasers, which is formed to execute the steps of the method according to claim 1.

16. The laser device according to claim 15, wherein the control device comprises
at least one storage device for at least temporary storage of at least one control dataset, wherein the control dataset or datasets include(s) control data for positioning and/or for focusing individual laser pulses in the polymer material; and
at least one beam device for beam guidance and/or beam shaping and/or beam deflection and/or beam focusing of a laser beam of the laser.

17. The laser device according to claim 16, wherein the laser device is formed as an eye surgical treatment apparatus.

18. A computer program including commands, which cause a laser device with at least one eye surgical laser and with at least one control device for the laser or lasers, to execute the method steps according to claim 1.

19. A non-transitory computer-readable medium, on which the computer program according to claim 18 is stored.

20. A method for performing a surgical procedure on a polymer material with a laser of a laser device comprising at least the following steps:
generating a plurality of first laser pulses with a first energy density within a preset energy range and below a photodisruption regime of a polymer material of a region of an optical element;
irradiating a core region of the region with the first laser pulses, wherein a refractive index of the polymer material changes at each irradiation spot irradiated with the first laser pulses depending thereon;
generating a plurality of first irradiation lines within the core region by means of a plurality of irradiation spots and thereby generating a first optical correction in the core region;
generating a plurality of second laser pulses with a second energy density within the energy range and below a photodisruption regime of the polymer material of the region of the optical element, wherein the second energy density is different from the first energy density;
irradiating an edge region of the region, which surrounds the core region at least in certain areas, with the second laser pulses, wherein the refractive index of the polymer material changes at each irradiation spot irradiated with the second laser pulses depending thereon; and
generating a plurality of second irradiation lines within the edge region by means of a plurality of irradiation spots and thereby generating a second optical correction different from the first optical correction in the edge region.

21. The method for performing a surgical procedure according to claim 20, wherein the second optical correction in the edge region is generated inferior than the first optical correction in the core region.

22. The method for performing a surgical procedure according to claim 20, wherein the first irradiation lines and/or the second irradiation lines are substantially annularly generated in the region.

23. The method for performing a surgical procedure according to claim 22, wherein the first irradiation lines and/or the second irradiation lines are generated concentrically to each other.

24. The method for performing a surgical procedure according to claim 20, wherein a transition from the core region to the edge region is preset depending on at least one parameter limiting the laser device.

25. The method for performing a surgical procedure according to claim 20, wherein the edge region is used as a transitional zone from the region to be treated to a region not to be treated.

26. The method for performing a surgical procedure according to claim 20, wherein a predefined correction of the optical element is performed in the core region.

27. The method for performing a surgical procedure according to claim 20, wherein at least the first energy density is adjusted depending on a respective distance of the irradiation spots to each other and/or depending on respective distances of the first irradiation lines to each other and/or depending on a laser pulse energy of the respective laser pulses.

28. The method for performing a surgical procedure according to claim 20, wherein an axicon shape of the optical region is generated by means of the method.

29. The method for performing a surgical procedure according to claim 20, wherein at least a height of the second irradiation line is generated differently than the height of the first irradiation lines and/or a distance between the second irradiation lines is generated differently than a distance of the first irradiation lines and/or the laser pulses are generated with a lower predefined second energy for the second irradiation lines than the laser pulses with the predefined first energy for the first irradiation lines for generating the second optical correction.

30. The method for performing a surgical procedure according to claim 20, wherein different second corrections are generated in the edge region from an inner edge, which faces the core region, to an outer edge, which faces away from the core region.

31. The method for performing a surgical procedure according to claim 20, wherein at the outer edge of the edge region, an inferior correction is generated than at the inner edge of the edge region.

32. The method for performing a surgical procedure according to claim 20, wherein the laser pulses are emitted in a wavelength range between 200 nm and 2 μm, in particular between 400 nm and 1450 nm, at a respective pulse duration between 1 fs and 1 ps, in particular between 10 fs and 100 fs, and a repetition frequency of greater than 10 kHz, in particular between 1 MHz and 100 MHz.

33. The method for performing a surgical procedure according to claim 20, wherein the control of the laser is effected such that topographic and/or pachymetric and/or morphologic data of a cornea as the polymer material is taken into account.
